(19) United States
(12) Patent Application Publication
(10) Pub. No.: US 2002/0040031 A1
(43) Pub. Date: Apr. 4, 2002

(54) Methods for prevention of accumulation of amyloid beta peptide in the central nervous system

(76) Inventors: Michelle S. Glasky, Irvine, CA (US); Debomoy K. Lahiri, Indianapolis, IN (US); Martin R. Farlow, Indianapolis, IN (US)

Correspondence Address: OPPENHEIMER WOLFF & DONNELLY LLP, 2029 Century Park East, Suite 3800, Los Angeles, CA, 90067, US

(21) Appl. No.: 09/899,611

(22) Filed: Jul. 5, 2001

Related U.S. Application Data
(60) Provisional application No. 60/216,845, filed on Jul. 7, 2000.

(51) Int. Cl. A61K 31/522; A61K 31/505; A61K 31/404

(52) U.S. Cl. 514/263; 514/256; 514/419

(57) ABSTRACT

A method of either inhibiting the formation of Aβ or stimulating the formation of sAPP comprises administering to a patient with a neurological disease or a patient at risk of developing a neurological disease an effective quantity of a purine derivative or analogue, a tetrahydroindolone derivative or analogue, or a pyrimidine derivative or analogue. If the compound is a purine derivative, the purine moiety can be guanine or hypoxanthine. The neurological disease can be a neurodegenerative disease such as Alzheimer's disease or a neurodevelopmental disorder such as Down's syndrome. Typically, the compound can pass through the blood-brain barrier. The purine moiety can be hypoxanthine or guanine. A particularly preferred purine derivative is N-4-carboxyphenyl-3-(6-oxohydropurin-9-yl) propanamide.

### CROSS-REFERENCES

This application claims priority from Provisional Application Ser. No. 60,216,845, filed Jul. 7, 2000, by Michelle S. Glasky, Debomoy K. Lahiri, and Martin R. Farlow, and entitled “Methods for Prevention of Accumulation of Amyloid Beta Peptide in The Central Nervous System by Treatment with Bifunctional Purine Analogues,” which is incorporated herein in its entirety by this reference.

### BACKGROUND OF THE INVENTION

This invention is directed to methods for blockage of accumulation of amyloid beta-peptide (Aβ) in patients with neurological diseases including neurodegenerative diseases such as Alzheimer's disease and neurodevelopmental disorders such as Down's syndrome, particularly with purine derivatives or analogues, pyrimidine derivatives or analogues, or tetrahydroindolone derivatives or analogues.

Alzheimer's disease (AD) is characterized by the cerebrovascular deposition of amyloid beta-peptide (Aβ) which is derived from a large integral membrane glycoprotein, β-amyloid precursor protein (APP). APP is processed by three proteases designated as α-, β-, and γ-secretases. The α-secretase cleaves APP within Aβ (between residues 16 and 17) to the secreted derivative sAPP and precludes Aβ formation. The processing of APP by (α-secretase is altered by growth factors and M1 and M3 cell surface receptors. These agents increase sAPP secretion and also reduce Aβ production in some cell types. The stimulation of sAPP secretion by growth factors is partly mediated by protein kinase C (PKC) and partly by tyrosine kinase activities. The growth factors that increase sAPP secretion include nerve growth factor (NGF) and basic fibroblast growth factor (bFGF). Purine derivatives, such as AIT-082, have been shown to stimulate secretion of neurotrophic growth factors.

Therefore, there exists a need for methods that can inhibit the formation of Aβ and can stimulate the formation of sAPP in patients with neurological diseases, including neurodegenerative diseases such as AD and neurodevelopmental disorders such as Down's syndrome. Preferably, these methods should be able to be combined with methods that enable active compounds to bypass the blood-brain barrier, making combined therapy more efficient. These methods should also be suitable for use with compounds or pharmaceutical compositions that can stimulate nerve growth or regeneration in patients neurological diseases, including neurodegenerative diseases such as AD and neurodevelopmental disorders such as Down's syndrome, thus reversing the course of the disease.

### SUMMARY

One embodiment of the present invention is a method of either inhibiting the formation of Aβ or stimulating the formation of sAPP by administering to a patient with a neurological disease or a patient at risk of developing a neurological disease an effective quantity of a compound comprising: (1) a moiety A selected from the group consisting of a purine moiety, a purine analogue, a tetrahydroindolone moiety, a tetrahydroindolone analogue, a pyrimidine moiety, and a pyrimidine analogue; (2) a hydrocarbyl moiety L of 1 to 6 carbon atoms that is linked to the moiety A and that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and (3) a moiety B that is linked to the moiety L though a carbonyl group wherein B is —OZ or N(Y₁)—D, where Z is hydrogen, alkyl, aryl, heteroaryl, cycloalkyl, aralkyl, or heteroaralkyl; D is a moiety that promotes absorption of the compound; and Y₁ is hydrogen, alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms, which can be N, O, or S.

The purine moiety can be selected from the group consisting of hypoxanthine and guanine, as well as other purine moieties. A number of purine derivatives suitable for use in methods according to the present invention are disclosed. A particularly preferred purine derivative is N-4-carboxyphenyl-3-(6-oxohydropurin-9-yl) propanamide. Preferably, the compound is capable of passing through the blood-brain barrier.

The neurological disease can be a neurodegenerative disease, such as, but not limited to, Alzheimer's disease (AD). Alternatively, the neurological disease can be a neurodevelopmental disorder such as, but not limited to, Down's syndrome.

### BRIEF DESCRIPTION OF THE DRAWINGS

The following invention will become better understood with reference to the specification, appended claims, and accompanying drawings, where:

FIG. 1 is a photograph of the transferred proteins of a gel electrophoresis (immunoblot) of proteins from PC12 cells in culture treated with NGF, bFGF, or the bifunctional purine derivative N-4-carboxyphenyl-3-(6-oxohydropurin-9-yl) propanamide (also known as AIT-082) probed with anti-APP antibody with immunodetection by an enzymatic color method; and

FIG. 2 is a graphical representation of the intensity of the bands of a Western immunoblot, similar to FIG. 1, as determined by densitometry scanning.

### DESCRIPTION

We have discovered that the bifunctional purine derivative N-4-carboxyphenyl-3-(6-oxohydropurin-9-yl) propanamide (also known as AIT-082 and leteprinim potassium), which bypasses the blood-brain barrier and is transported into brain by a nonsaturable mechanism, can act to increase the secretion of sAPP and therefore to decrease the formation of Aβ. This property of increasing the secretion of sAPP and decreasing the formation of Aβ, therefore, should also be possessed by other bifunctional purine analogues, as discussed below, as well as other compounds, including tetrahydrolone derivatives and analogues, and pyrimidine derivatives and analogues.

Therefore, in general, a method according to the present invention is a method of either inhibiting the formation of Aβ or stimulating the formation of sAPP comprising administering to a patient with a neurological disease or a patient at risk of developing a neurological disease an effective amount of a compound having the activity of either inhibiting the formation of Aβ or stimulating the formation of sAPP, the compound comprising: (1) a moiety A selected from the group consisting of a purine moiety, a purine analogue, a tetrahydroindolone moiety, a tetrahydroindolone analogue, a pyrimidine moiety, and a pyrimidine analogue; (2) a hydrocarbyl moiety L of 1 to 6 carbon atoms that is linked to the moiety A and that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and (3) a moiety B that is linked to the moiety L through a carbonyl group wherein B is —OZ or N(Y₁)—D, where Z is hydrogen, alkyl, aryl, heteroaryl, cycloalkyl, aralkyl, or heteroaralkyl; D is a moiety that promotes absorption of the compound having the activity of either inhibiting the formation of Aβ or stimulating the formation of sAPP; and Y₁ is hydrogen, alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms, which can be N, O, or S.

Typically, a compound useful in a method of the present invention is capable of passing through the blood-brain barrier.

In one preferred embodiment of methods according to the present invention, the moiety A is a purine moiety.

In one alternative, A is a substituted or unsubstituted hypoxanthine moiety. Typically, in this alternative, L has the structure —(CH₂)ₙ—where n is an integer from 1 to 6.

The compound having the activity of either inhibiting the formation of Aβ or stimulating the formation of sAPP can be a compound of formula (I)

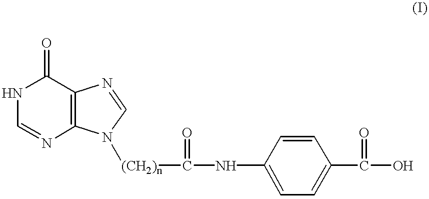

where n is an integer from 1 to 6 and R is hydrogen or lower alkyl or is a salt or prodrug ester of a compound of formula (I) wherein n is an integer from 1 to 6 and R is hydrogen or lower alkyl. Typically, the compound is a compound of formula (I) wherein n is an integer from 1 to 6 and R is hydrogen or lower alkyl. Typically, R is hydrogen, and the compound is N-4-[[3-(6-oxo-1,6-dihydropurin-9-yl)-1-oxopropyl]amino]benzoic acid, designated AIT-082. Alternatively, R is ethyl, and the compound is N-4-[[3-(6-oxo-1,6-dihydropurin-9-yl)-1-oxopropyl]amino]benzoic acid ethyl ester.

When the purine moiety is hypoxanthine, a preferred purine derivative is a compound of formula (I)

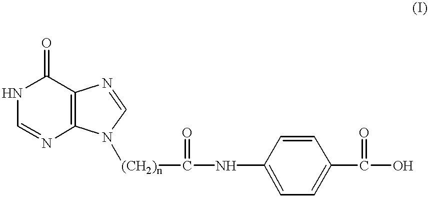

wherein n is an integer from 1 to 6 or of a salt or prodrug ester of formula (I) wherein n is an integer from 1 to 6. Typically, the purine derivative is a compound of formula (I) wherein n is an integer from 1 to 6. Preferably, n is 2 and the compound is N-4-carboxyphenyl-3-(6-oxohydropurin-9-yl) propanamide, also known as AIT-082. The activity of this compound is described further in the Example.

Alternatively, the purine derivative can be a 9-substituted hypoxanthine derivative of formula (II)

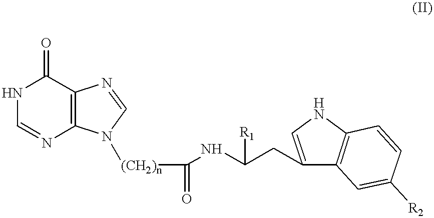

wherein n is a integer from 1 to 6, R₁ is selected from the group consisting of H, COOH, and COOW₁, where W₁ is selected from the group consisting of lower alkyl, amino, and lower alkylamino, and R₂ is selected from the group consisting of H and OH.

In this alternative, for one particularly preferred purine derivative, n is 2, R₁ is H and R₂ is OH and the purine derivative is N-(2-(5-hydroxyindol-3-yl))ethyl-3-(6-oxohydropurine-9-yl) propanamide. In this alternative, for another particularly preferred purine derivative, n is 2, R₁ is H and R₂ is H and the purine derivative is N-(2-indol-3-yl) ethyl-3-(6-oxohydropurin-9-yl) propanamide. In this alternative, for still another particularly preferred purine derivative, n is 2, R₁ is COOH, and R₂ is OH and the purine derivative is N-(1-carboxyl-(2-(5-hydroxyindol-3-yl))ethyl-3-(6-oxohydropurin-9-yl) propanamide.

As another alternative, the purine derivative can be a 9-substituted hypoxanthine derivative of formula (III)

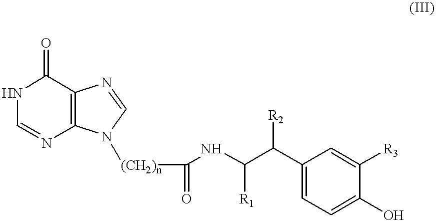

wherein n is an integer from 1 to 6, R₁ is selected from the group consisting of H, COOH, and COOW₁, wherein W₁ is selected from the group consisting of lower alkyl, amino, and lower alkylamino, R₂ is selected from the group consisting of H and OH, and R₃ is selected from the group consisting of H and OH.

In this alternative, for one particularly preferred purine derivative, n is 2, R₁ is H, R₂ is H, and R₃ is OH, and the purine derivative is N-(2-(3,4-dihydroxyphenyl))ethyl-3-(6-oxohydropurin-9-yl) propanamide. In this alternative, for another particularly preferred purine derivative, n is 2, R₁ is H, R₂ is OH, and R₃ is OH, and the purine derivative is N-(2-hydroxy-2-(3,4-dihydroxyphenyl))ethyl-3-(6-oxohydropurin-9-yl) propanamide. In this alternative, for still another particularly preferred purine derivative, n is 2, R₁ is COOH, R₂ is H, and R₃ is OH, and the purine derivative is N-(1-carboxyl-2-(3,4-dihydroxyphenyl))ethyl-3-(6-oxohydropurin-9-yl) propanamide.

When the purine moiety is guanine, one preferred purine derivative is a 9-substituted guanine derivative of formula (IV)

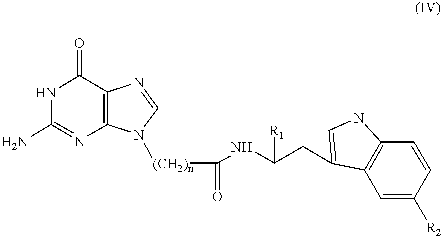

wherein n is an integer from 1 to 6, R, is selected from the group consisting of H, COOH, and COOW₁, or W₁ is lower alkyl, amino, or lower alkylamino, and R₂ is selected from the group consisting of H and OH.

In this alternative, for one particularly preferred purine derivative, n is 2, R₁ is H, and R₂ is OH, and the purine derivative is N-(2-(5-hydroxindol-3-yl))ethyl-3-(2-amino-6-oxohydropurin-9-yl) propanamide. In this alternative, for another particularly preferred purine derivative, n is 2, R₁ is H, and R₂ is H and the purine derivative is N-(2-(2-indol-3-yl)ethyl))-3-(2-amino-6-oxohydropurin-9-yl)) propanamide. In this alternative, for still another particularly preferred purine derivative, n is 2, R₁ is COOH, and R₂ is OH, and the purine derivative is N-(1 -carboxyl)-(2-(5-hydroxyindol-3-yl))ethyl-3-(2-amino-6-oxohydropurin-9-yl) propanamide.

Alternatively, the purine derivative can be a 9-substituted guanine derivative of formula (V) wherein n is an integer from 1 to 6.

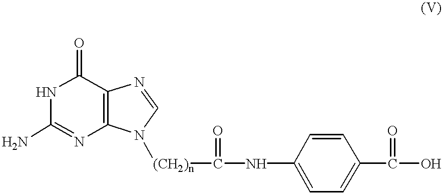

In this alternative, for one particularly preferred purine derivative, n is 2 and the compound is N-4-carboxyphenyl-3-(2-amino-6-oxohydropurin-9-yl) propanamide.

Alternatively, the purine derivative can be a 9-substituted guanine derivative of formula (VI) wherein n is an integer from 1 to 6.

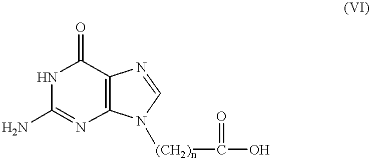

In this alternative, for one particularly preferred purine derivative, n is 2 and the compound is 3-(2-amino-6-oxohydropurine-9-yl) propanoic acid.

Alternatively, the purine derivative can be a 9-substituted guanine derivative of formula (VII) wherein n is an in integer from 1 to 6, p is an integer from 1 to 6, and q is an integer from 1 to 3.

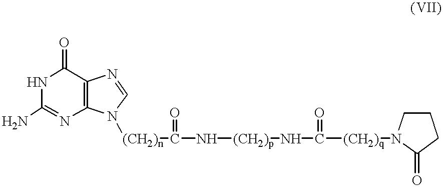

In this alternative, for one particularly preferred purine derivative, n is 2, p is 2, and q is 1, and the purine derivative is N-[2-[[2-(2-oxopyrrolidin-1-yl)-1-oxoethyl]amino]ethyl]propanamide.

Alternatively, the purine derivative can be a 9-substituted guanine derivative of formula (VIII) wherein R₁ is selected from the group consisting of H, COOH, and COOW₁, where W₁ is selected from the group consisting of lower alkyl, amino, and lower alkylamino, R₂ is selected from the group consisting of H and OH, and R₃ is selected from the group consisting of H and OH.

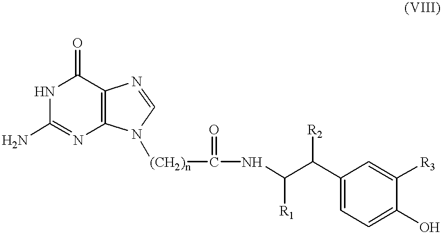

In this alternative, for one particularly preferred purine derivative, n is 2, R₁ is H, R₂ is H, and R₃ is OH, and the purine derivative is N-(2-(3,4-dihydroxyphenyl)ethyl-3-(2-amino-6-oxohydropurin-9-yl) propanamide. In this alternative, for another particularly preferred purine derivative, n is 2, R₁ is H, R₂ is OH, and R₃ is OH, and the purine derivative is N-(2-hydroxy-2-(3,4-dihydroxyphenyl)ethyl)-3-(2-amino-6-oxohydropurin-9-yl) propanamide. In this alternative, for still another particularly preferred purine derivative, n is 2, R₁ is COOH, R₂ is H, and R₃ is H and the compound is N-(1-carboxyl-2-(3,4-dihydroxyphenyl)ethyl)-3-(2-amino-6-oxohydropurin-9-yl) propanamide.

Alternatively, the purine derivative can be a 9-substituted guanine derivative of formula (IX) wherein n is an integer from 1 to 6 and p is an integer from 1 to 3.

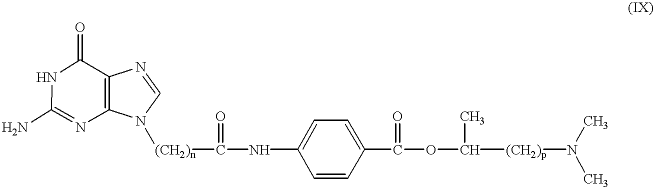

In this alternative, for one particularly preferred purine derivative, n is 2, p is 1, and the compound is the 1-(dimethylamino)-2-propyl ester of N-4-carboxyphenyl-3-(2-amino-6-oxohydropurin-9-yl) propanamide.

Other bifunctional hypoxanthine derivatives suitable for use in methods according to the present invention are disclosed in U.S. Pat. No. 5,091,432 to Glasky, incorporated herein by this reference. Other bifunctional guanine derivatives suitable for use in methods according to the present invention are disclosed in U.S. patent application Ser. No. 09/419,153, by Glasky et al., incorporated herein by this reference.

More generally, purine-based compounds suitable for use in methods according to the present invention are compounds in which A is a substituted or unsubstituted 9-atom bicyclic moiety in which the 5-membered ring has 1 to 3 nitrogen atoms, the bicyclic moiety having the structure of formula (X)

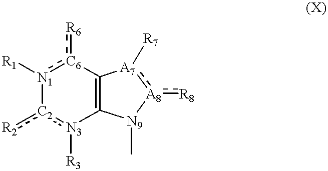

where:

(1) if the bond between N₁ and the bond between C₅ is a single bond, then the bond between C₆ and R₆ is a double bond, R₆ is O or S, and R₁ is hydrogen, alkyl, aralkyl, cycloalkyl, or heteroaralkyl;

(2) if the bond between N₁ and C₆ is a double bond, then the bond between C₆ and R₆ is a single bond, R₁ is not present, and R₆ is hydrogen, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(3) if the bond between C₂ and N₃ is a single bond, then the bond between C₂ and R₂ is a double bond, R₂ is O or S, and R₃ is hydrogen or alkyl;

(4) if the bond between C₂ and N₃ is a double bond, then the bond between C₂ is a single bond, R₃ is not present, and R₂ is hydrogen, alkyl, aralkyl, cycloalkyl, heteroaralkyl, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(5) A₇ and A₈ are C or N;

(a) if A₇ and A₈ are both C and the bond between A₇ and A₈ is a single bond, then the bond between A₈ and R₈ is two single bonds to two hydrogen atoms or is a double bond in which R₈ is O, or S and R₇ is two hydrogen atoms;

(b) if A₇ and A₈ are both C and the bond between A₇ and A8 is a double bond, then R₇ is hydrogen, the bond between A₈ and R₈ is a single bond and R₈ is hydrogen, halo, alkyl, alkenyl, aryl, aralkyl, aralkenyl, heteroaryl, heteroaralkyl, or heteroaralkenyl;

(c) if A₇ and A₈ are both N, then the bond between A₇ and A₈ is a double bond, and R₇ and R₈ are not present;

(d) if A₇ is C and A₈ is N, then the bond between A₇ and A₈ is a double bond, R₇ is hydrogen, and R₈ is not present;

(e) if A₇ is N, A₈ is C, and the bond between A₇ and A₈ is a double bond, then R₇ is not present, the bond between A₈ is a single bond, and R₈ is hydrogen, halo, alkyl, alkenyl, aryl, aralkyl, aralkenyl, heteroaryl, heteroaralkyl, or heteroaralkenyl;

(f) if A₇ is N, A₈ is C, and the bond between A₇ and A₈ is a single bond, then R₇ is hydrogen, alkyl, aryl, aralkyl, heteroaryl, or heteroaralkyl, the bond between A₈ and R₈ is a double bond, and R₈ is O or S; and

(6) N₉ is bonded to L; with the proviso that A does not have the structure of an unsubstituted guanine or hypoxanthine.

The purine moiety can be a purine moiety of formula (XI)

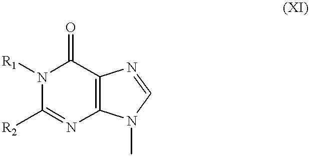

in which:

(1) R₁ is selected from the group consisting of hydrogen, alkyl, aralkyl, cycloalkyl, and heteroaralkyl; and

(2) R₂ is selected from the group consisting of hydrogen, alkyl, aralkyl, cycloalkyl, heteroaralkyl, halo, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, arylkoxycarbonyl, heteroarylokoxycarbonyl, alkylaminocarbonyl, arylaminocarbonyl, heteroarylaminocarbonyl, aralkylaminocarbonyl, or heteroarylkylaminocarbonyl in which the alkyl portions could be cyclic and can contain from one to three heteroatoms which could be N, O, or S, with the proviso that both R₁ and R₂ are not hydrogen and that R₁ is not hydrogen when R₂ is amino.

The purine moiety of formula (XI) is a hypoxanthine or a guanine derivative but excludes unsubstituted hypoxanthine, in which R₁ and R₂ are hydrogen, and unsubstituted guanine, in which R₁ is hydrogen and R₂ is amino.

In one particularly preferred embodiment, R₁ is butyl and R₂ is hydrogen.

In another preferred embodiment, R₁ is benzyl and R₂ is hydrogen.

In another preferred embodiment, R₁ is dimethylaminoethyl and R₂ is hydrogen.

In another preferred embodiment, R₁ is cyclopentyl and R₂ is hydrogen.

In another preferred embodiment, R₁ is cyclohexylmethyl and R₂ is hydrogen.

In another preferred embodiment, R₁ is cyclopropylmethyl and R₂ is hydrogen.

In another preferred embodiment, R₁ is hydrogen and R₂ is phenyl.

In another preferred embodiment, R₁ is hydrogen and R₂ is trifluoromethyl.

In another preferred embodiment, R₁ is hydrogen and R₂ is butyl.

In another preferred embodiment, R₁ is butyl and R₂ is butyl.

In another preferred embodiment, R₁ is hydrogen and R₂ is methyl.

In another preferred embodiment, R₁ is hydrogen and R₂ is phenylamino.

Alternatively, the purine moiety can be a purine moiety of Formula (XII)

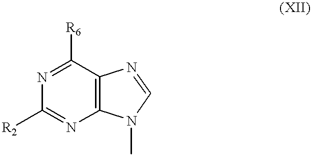

in which:

(1) R₂ is selected from the group consisting of hydrogen, halo, amino, OQ₃, SQ₃, NHNH₂, NHOQ₃, NQ₃Q₄, or NHQ₃, where Q₃ and Q₄ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N; O, or S, and when Q₃ and Q₄ are present together and are alkyl, they can be taken together to form a 5- or 6-membered ring which can contain one other heteroatom which can be N, O, or S, of which the N can be further substituted with Y₃ where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaryloxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S; and

(2) R₆ is selected from the group consisting of hydrogen, halo, amino, OQ₅, SQ₅, NHNH₂, NHOQ₅, NQ₅Q₆, or NHQ₆, where Q₅ and Q₆ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N, O, or S, and when Q₅ and Q₆ are present together and are alkyl, they can be taken together to form a 5- or 6- membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, heteroarylsulfonyl, aralkylsulfonyl, heteroaralkylsulfonyl, alkoxycarbonyl, aryloxycarbonyl, heteroaryloxycarbonyl, arylkoxycarbonyl, heteroarylkoxycarbonyl, alkylaminocarbonyl, arylaminocarbonyl, heteroarylaminocarbonyl, aralkylaminocarbonyl, or heteroaralkylaminocarbonyl in which the alkyl portions can be cyclic and can contain from 1 to 3 heteroatoms which can be N, O, or S.

In one preferred example of this embodiment, R₂ is hydrogen and R₆ is —NH₂ or —N(CH₃)₂.

In another preferred example of this embodiment, R₂ is hydrogen and R₆ is Cl.

In yet another preferred example of this embodiment, R₂ is —NH₂ and R₆ is Cl.

In another alternative, the purine moiety is the purine moiety of Formula (XIII)

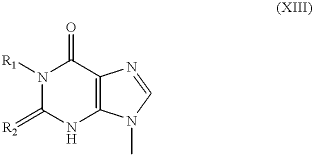

in which:

(1) R₁ is hydrogen, alkyl, aralkyl, cycloalkyl, or heteroaralkyl; and

(2) R₂ is O or S.

Preferably, in this embodiment, R₁ is hydrogen and R₂ is O or S.

Particularly preferred purine-based compounds for use in methods according to the present invention include: (1) 4-[3-(1-benzyl-6-oxo-1,6-dihydropurin-9-yl)propionylamino]benzoic acid ethyl ester; (2) 4-[3-(1-butyl-6-oxo-1,6-dihydropurin-9-yl) propionylamino]benzoic acid ethyl ester; (3) 4-[3-(1-methyl-6-oxo-1,6-dihydropurin-9-yl) propionylamino]benzoic acid ethyl ester; (4) 4-[3-(1-(2-dimethylaminoethyl)-6-oxo-1,6-dihydropurin-9-yl)propionylamino]benzoic acid ethyl ester; (5) 4-[3-(2,6-dioxo-1,2,3,6-tetrahydropurin-9-yl)propionylamino]benzoic acid ethyl ester; (6) 4-[3-(6-methoxypurin-9-yl)propionylamino]benzoic acid ethyl ester; (7) 4-[3-(6-dimethylaminopurin-9-yl)propionylamino]benzoic acid ethyl ester; (8) 4-[3-(2-amino-6-chloropurin-9-yl)propionylamino]benzoic acid ethyl ester; (9) 4-[2-(6-oxo-2-thioxo-1,2,3,6-tetrahydropurin-9-yl)propionylamino]benzoic acid ethyl ester; (10) 4-[2-(2-butyl-6-oxo-1,6-dihydropurin-9-yl)propionylamino]benzoic acid ethyl ester; (11) 4-[2-(6-oxo-2-phenyl-1,6-dihydropurin-9-yl)propionylamino]benzoic acid ethyl ester; (12) 4-{[3-(6-chloropurin-9-yl)propionyl]methylamino}benzoic acid methyl ester; (13) 3-(1-benzyl-6-oxo-1,6-dihydropurin-9-yl)-N-[3-(2-oxopyrrolidin-1-yl)propyl]propionamide; (14) 3-(1-benzyl-6-oxo-1,6-dihydropurin-9-yl)-N-{2-[2-(2-oxopyrrolidin-1 -yl)acetylamino]ethyl}propionamide; (15) N-3-(2-oxopyrrolidin-1-yl)propyl]-3-(6-oxo-2-thioxo-1,2,3,6-tetrahydropurin-9-yl) propionamide; and (16) 3-(l-benzyl-6-oxo-1,6-dihydropurin-9-yl)-N-(3-morpholin-4-yl-propyl) propionamide.

In another alternative of methods according to the present invention, the compound is a tetrahydroindolone derivative or analogue where A is a 9-atom bicyclic moiety in which the 5-membered ring has one to three nitrogen atoms, the bicyclic moiety having the structure of formula (XIV)

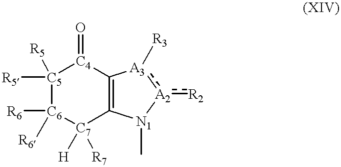

where:

(1) N₁ is bonded to L;

(2) A₂and A₃are C or N;

(a) If A₂ and A₃ are both C and the bond between A₂ and A₃ is a single bond, then the bond between A₂ and R₂ is two single bonds, two hydrogen atoms or is a double bond in which R₂ is O or S and R₃ is two hydrogen atoms;

(b) If A₂ and A₃ are both C and the bond between A₂ and A₃ is a double bond, then R₃ is hydrogen, the bond between A₂ and R₂ is a single bond and R₂ is hydrogen, halo, alkyl, alkenyl, aryl, aralkyl, aralkenyl, heteroaryl, heteroaralkyl, or heteroaralkenyl;

(c) If A₂ and A₃ are both N, then the bond between A₂ and A₃ is a double bond and R₂ and R₃ are not present;

(d) If A₂ is N and A₃ is C, then the bond between A₂ and A₃ is a double bond, R₂ is not present, and R₃ is hydrogen;

(e) If A₂ is C, A₃ is N, and the bond between A₂ and A₃ is a double bond, then R₃ is not present, the bond between A₂ and R₂ is a single bond, and R₂ is hydrogen, halo, alkyl, alkenyl, aryl, aralkyl, aralkenyl, heteroaryl, heteroaralkyl, or heteroaralkenyl;

(f) If A₂ is C, A₃ is N, and the bond between A₂ and A₃ is a single bond, then R₃ is hydrogen, alkyl, aryl, aralkyl, heteroaryl, or heteroaralkenyl, the bond between A₂ and R₂ is a double bond, and A₂ is O or S;

(3) R₅ is hydrogen, alkyl, aryl, aralkyl, heteroaryl, heteroaralkyl, alkanoyl, aroyl, heteroaroyl, aralkanoyl, heteroaralkanoyl, NH₂, NHQ₁, NQ₁Q₂, OH, OQ₁, or SQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom, which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(4) R_{5′} is hydrogen unless R₅ is alkyl, in which case R₅ is hydrogen or the same alkyl as R₅;

(5) R₅ and R_{5′} can be taken together as a double bond to C₅, and can be O, S, NQ₃, or C which can be substituted with one or two groups R₅, where Q₃ is alkyl, aralkyl, heteroaralkyl, aryl, heteroaryl, alkanoyl, aroyl, aralkanoyl, heteroaralkanoyl, or heteroaroyl, in which the alkyl portions can be cyclic and can contain from 1 to 3 heteroatoms which can be N, O, or S;

(6) R₆ is hydrogen, alkyl, aryl, aralkyl, heteroaryl, heteroaralkyl, NH₂, NHQ₄, NQ₄Q₅, OH, OQ₄, or SQ₄, where Q₄ and Q₅ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₄ and Q₅ are present together and are alkyl, they can be taken together to form a 5- or 6-membered ring which can contain one other heteroatom, which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(7) R_{6′} is hydrogen unless R₆ is alkyl, in which case R_{6′} is hydrogen or the same alkyl as R₆;

(8) R₆ and R_{6′} can be taken together as a double bond to C₆ and can be O, S, NQ₆, or C which can be substituted with one or two groups R₅, and where Q₆ is alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S; and

(9) R₇ is hydrogen unless R₅ is alkyl and R_{5′} is hydrogen, in which case R₇ is the same alkyl as R₅.

Typically, A is a tetrahydroindolone moiety. More typically, the tetrahydroindolone moiety is a tetrahydroindolone moiety of formula (XV)

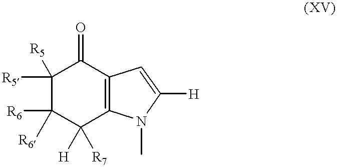

in which:

(1) R₅ is hydrogen, alkyl, aryl, aralkyl, heteroaryl, heteroaralkyl, alkanoyl, aroyl, heteroaroyl, aralkanoyl, heteroaralkanoyl, NH₂, NH₁, NQ₁Q₂, OH, OQ₁, or SQ₁, where Q₁ and Q₂ are alkyl, aralkyl, heteroaralkyl, aryl, heteroaryl, alkanoyl, aroyl, aralkanoyl, heteroaralkanoyl, or heteroaroyl, in which the alkyl portions can be cyclic and can contain from one to three heteroatoms which can be N, O, or S;

(2) R_{5′} is hydrogen;

(3) R₆ is hydrogen, alkyl, aryl, aralkyl, heteroaryl, heteroaralkyl, alkanoyl, aroyl, heteroaroyl, aralkanoyl, heteroaralkanoyl, NH₂, NHW₁, NQ₁Q₂, OH, OQ₁, or SQ₁, where Q₁ and Q₂ are aralkyl, heteroaralkyl, aryl, heteroaryl, alkanoyl, aroyl, aralkanoyl, heteroaralkanoyl, or heteroaroyl, in which the alkyl portions can be cyclic and can contain from one to three heteroatoms which can be N, O, or S and where W₁ is alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from one to three heteroatoms which can be N, O, or S;

(4) R_{6′} is hydrogen; and

(5) R₇ is hydrogen.

Typically, R₅, R_{5′}, R₆, R_{6′}, and R₇ are all hydrogen.

When A is a tetrahydroindolone moiety, preferred compounds are 4-[3-(4-oxo-4,5,6,7-tetrahydroindolon-1-yl) propionylamino]benzoic acid ethyl ester and 4-[3-(4-oxo-4,5,6,7-tetrahydroindolon-1 -yl) propionylamino]benzoic acid.

In another alternative, the compound is a pyrimidine derivative or pyrimidine analogue. In this alternative, A is is an amino-substituted 6-membered heterocyclic moiety of formula (XVI)

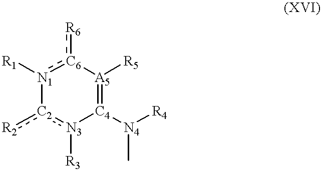

where:

(1) if the bond between N₁ and the bond between C₆ is a single bond, then the bond between C₆ and R₆ is a double bond, R₆ is O or S, and R₁ is hydrogen, alkyl, aralkyl, cycloalkyl, or heteroaralkyl;

(2) if the bond between N₁ and C₆ is a double bond, then the bond between C₆ and R₆ is a single bond, R₁ is not present, and R₆ is hydrogen, halo, amino, OH, OQ₁, SQ₁, NHNH₂, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(3) if the bond between C₂ and N₃ is a single bond, then the bond between C₂ and R₂ is a double bond, R₂ is O or S, and R₃ is hydrogen or alkyl;

(4) if the bond between C₂ and N₃ is a double bond, then the bond between C₂ and R₂ is a single bond, R₃ is not present, and R₂ is hydrogen, alkyl, aralkyl, cycloalkyl, heteroaralkyl, halo, amino, OH, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₃, where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(5) R₄ is hydrogen, alkyl, aralkyl, heteroaralkyl, alkanoyl, aroyl, heteroaroyl, aralkanoyl, heteroaralkanoyl, alkylsulfonyl, arylsulfonyl, heteroarylsulfonyl, alkoxycarbonyl, aryloxycarbonyl, heteroaryloxycarbonyl, alkylaminocarbonyl, arylaminocarbonyl, or heteroarylaminocarbonyl;

(6) A₅ is carbon or nitrogen;

(7) if A₅ is nitrogen, then R₅ is not present;

(8) if A₅ is carbon, then R₅ is hydrogen, amino, alkyl, alkoxy, halo, nitro, aryl, cyano, alkenyl, or alkaryl;

(9) if R₅ and R₆ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S; and

(10) N₄ is bonded to L.

Typically, A₅ is carbon and the 6-membered heterocyclic moiety is a pyrimidine moiety.

When A is a pyrimidine moiety, in one alternative, R₂ is O and R₃ is hydrogen. In this alternative, the pyrimidine moiety can be cytosine, thymine, uracil, 3-methyluracil, 3-methylthymine, 4-methylcytosine, 5-methylcytosine, 5-hydroxymethylcytosine, 5-hydroxyuracil, 5-carboxymethyluracil, or 5-hydroxymethyluracil.

In another alternative, R₂ is S and R₃ is hydrogen. In this alternative, the pyrimidine moiety can be 2-thiouracil, 5-methylamino-2-thiouracil, 5-methyl-2-thiouracil, or 2-thiocytosine.

In still another alternative, R₂ is amino and the bond between C₂ and N₃ is a double bond. In this alternative, the pyrimidine moiety can be 2-aminopyrimidinone or 2-amino-4-chloropyrimidine.

In still another alternative, R₂ is hydrogen and the bond between C₂ and N₃ is a double bond. In this alternative, the pyrimidine moiety can be 4-chloropyrimidine, 5-amino-4-chloropyrimidine, 4-chloro-5-methylpyrimidine, 4-chloro-5-hydroxymethylpyrimidine, or 4-chloro-5-carboxymethylpyrimidine.

In still another alternative, R₁ is hydrogen, methyl, or ethyl, R₅ is hydrogen, methyl, or ethyl, and R₆ is O. In this alternative, the pyrimidine moiety can be pyrimidinone.

Particularly preferred pyrimidine compounds include: 4-[3-(2-amino-6-chloropyrimidin-4-ylamino) propionylamino]benzoic acid ethyl ester; 4-[3-(5-amino-6-chloropyrimidin-4-ylamino) propionylamino]benzoic acid ethyl ester; 4-[3-(6-chloropyrimidin-4-ylamino) propionylamino]benzoic acid ethyl ester; 4-[3-(2-amino-6-chloropyrimidin-4-ylamino) propionylamino]benzoic acid; 4-[3-(6-chloropyrimidin-4-ylamino) propionylamino]benzoic acid; 4-[3-(5-amino-6-chloropyrimidin-4-ylamino)propionylamino]benzoic acid; 3-[3-(2-amino-6-chloropyrimidin-4-ylamino)propionylamino]benzoic acid ethyl ester; 3-[3-(6-chloropyrimidin-4-ylamino)propionylamino]benzoic acid ethyl ester; 3-[3-(5-amino-6-chloropyrimidin-4-ylamino)propionylamino]benzoic acid ethyl ester; 3-[3-(2-amino-6-chloropyrimidin-4-ylamino)propionylamino]benzoic acid; 3-[3-(6-chloropyrimidin-4-ylamino) propionylamino]benzoic acid; and 3-[3-(5-amino-6-chloropyrimidin-4-ylamino) propionylamino]benzoic acid.

In accordance with the present invention, and as used herein, the following terms, when appearing alone or as part of a moiety including other atoms or groups, are defined with the following meanings, unless explicitly stated otherwise. In addition, all groups described herein can be optionally substituted unless such substitution is excluded. The term “alkyl,” as used herein at all occurrences, refers to saturated aliphatic groups including straight-chain, branched-chain, and cyclic groups, all of which can be optionally substituted. Preferred alkyl groups contain 1 to 10 carbon atoms. Suitable alkyl groups include methyl, ethyl, and the like, and can be optionally substituted. The term “alkenyl,” as used herein at all occurrences, refers to unsaturated groups which contain at least one carbon-carbon double bond and includes straight-chain, branched-chain, and cyclic groups, all of which can be optionally substituted. Preferable alkenyl groups have 2 to 10 carbon atoms. The term “alkoxy” refers to the ether —O-alkyl, where alkyl is defined as as above. The term “aryl” refers to aromatic groups which have at least one ring having a conjugated π-electron system and includes carbocyclic aryl and biaryl, both of which may be optionally substituted. Preferred aryl groups have 6 to 10 carbon atoms. The term “aralkyl” refers to an alkyl group substituted with an aryl group. Suitable aralkyl groups include benzyl and the like; these groups can be optionally substituted. The term “aralkenyl” refers to an alkenyl group substituted with an aryl group. The term “heteroaryl” refers to carbon-containing 5-14 membered cyclic unsaturated radicals containing one, two, three, or four O, N, or S heteroatoms and having 6, 10, or 14π-electrons delocalized in one or more rings, e.g., pyridine, oxazole, indole, thiazole, isoxazole, pyrazole, pyrrole, each of which can be optionally substituted as discussed above. The term “sulfonyl” refers to the group —S(O₂)—. The term “alkanoyl” refers to the group —C(O)Rg, where Rg is alkyl. The term “aroyl” refers to the group —C(O)Rg, where Rg is aryl. Similar compound radicals involving a carbonyl group and other groups are defined by analogy. The term “aminocarbonyl” refers to the group —NHC(O)—. The term “oxycarbonyl” refers to the group —OC(O)—. The term “heteroaralkyl” refers to an alkyl group substituted with a heteroaryl group. Similarly, the term “heteroaralkenyl” refers to an alkenyl group substituted with a heteroaryl group. As used herein, the term “lower,” in reference to an alkyl or the alkyl portion of an another group including alkyl, is defined as a group containing one to six carbon atoms. The term “optionally substituted” refers to one or more substituents that can be lower alkyl, aryl, amino, hydroxy, lower alkoxy, aryloxy, lower alkylamino, arylamino, lower alkylthio, arylthio, or oxo, in some cases, other groups can be included, such as cyano, acetoxy, or halo. The term “halo” refers generally to fluoro, chloro, bromo, or iodo; more typically, “halo” refers to chloro.

As indicated above, the linker L is a hydrocarbyl moiety of 1 to 6 carbon atoms that can be cyclic, with the hydrocarbyl moiety being optionally substituted with one or more substituents selected from the group consisting of lower alkyl, amino, hydroxy, lower alkoxy, lower alkylamino, lower alkylthio, and oxo. Preferably, the linker L has the structure —(CH₂)ₙ—wherein n is an integer from 1 to 6. As detailed below, for most preferred embodiments of compounds useful in methods according to the present invention, a preferred linker has n equal to 2 or 3.

The moiety B is either: (i) —OZ, where Z is hydrogen, alkyl, aryl, heteroaryl, cycloalkyl, aralkyl, or heteroaralkyl; or (ii) N(Y₁)—D, where D is a moiety that promotes absorption of the compound, and Y₁ is hydrogen, alkyl, aryl, heteroaryl, aralkyl, heteroaralkyl, which, when taken with D, can form a cyclic 5- or 6-membered saturated ring which can contain one other heteroatom which can be O, N, or S, of which N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, heteroaralkylaminocarbonyl, in which the alkyl portions can be cyclic and can contain from 1 to 3 heteroatoms which can be N, O, or S. Typically, Y₁ is hydrogen. Where the moiety B is —OZ, the moiety B is a carboxylic acid or carboxylic acid or ester. Typically, where B is a carboxylic acid ester, the moiety Z is a lower alkyl, such as methyl, ethyl, butyl, propyl, or isopropyl.

In one alternative, the moiety D, as described above, is a moiety having at least one polar, charged, or hydrogen-bond-forming group to improve the metabolic and bioavailability properties of the compound. The moiety D can be, but is not limited to, a moiety with physiological or biological activity such as nootropic activity. In one alternative, the moiety D can be a moiety containing at least one carboxyl, carboxamide, carboxyl ester, or carbonyl function. In another alternative, the moiety D can be a moiety containing at least one hydroxyl, primary amino, secondary amino, tertiary amino, sulfhydryl, or sulfonamidyl function. The moiety D can be cyclic or acyclic. Preferred examples of the moiety D are described below.

When the moiety D is a cyclic or acyclic moiety containing at least one carbonyl, carboxamide, carboxyl ester, or carbonyl function, in one preferred example, D is a carboxylic acid or carboxylic acid ester with the structure

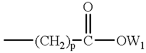

wherein p is an integer from 1 to 6 and W₁ is selected from the group consisting of hydrogen and lower alkyl. Typically, if W₁ is lower alkyl, it is methyl, ethyl, propyl, butyl, or isobutyl. Typically, p is 3. Typically, W₁ is hydrogen or ethyl.

In another preferred example, D and Y₁ are taken together to form a piperazine derivative as described in D. Manetti et al., “Molecular Simplification of 1,4-Diazabicyclo[4.3.0]nonan-9-ones Gives Piperazine Derivatives That Maintain High Nootropic Activity,” J. Med. Chem. 43: 4499-4507 (“Manetti et al. (2000)”). B is an analogue of structure

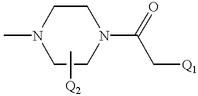

wherein Q₁ is hydrogen, methyl, ethyl, butyl, or propyl, Q₂ is hydrogen or methyl, where, if Q₂ is methyl, it can be located at either of the two possible positions in the piperazine ring.

In another preferred example, D has the structure

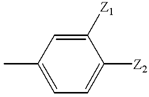

where one of Z₁ and Z₂ is hydrogen, and the other of Z₁ and Z₂ is —COOH or —COOW₁, wherein W₁ is alkyl. Typically, W₁ is selected from the group consisting of methyl, ethyl, propyl, butyl, and isobutyl. Either of Z₁ or Z₂ can be hydrogen. When Z, is hydrogen and Z₂ is —COOH, the moiety B is p-aminobenzoic acid (PABA). When Z₁ is —COOH and Z₂ is hydrogen, the moiety B is m-aminobenzoic acid (MABA). When Z₁ is hydrogen and Z₂ is —COOW₁, the moiety B is an ester of p-aminobenzoic acid (PABA). When Z₁ is —COOW₁ and Z₂ is hydrogen, the moiety B is an ester of m-aminobenzoic acid (MABA). Typically, these esters are ethyl esters.

When the moiety D is a moiety that contains at least one hydroxyl, primary amino, secondary amino, tertiary amino, sulfhydryl, or sufonamidyl function, in one preferred example, D is a phenylsulfonamidyl moiety of structure

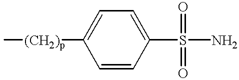

wherein p is an integer from 0 to 6. Typically, p is 2.

In another preferred example, D is an alkylpyridyl moiety of structure

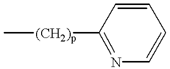

wherein p is an integer from 1 to 6. Typically, p is 1.

In another preferred example, D is a dialkylaminoalkyl moiety of the structure

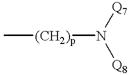

wherein p is an integer from 1 to 6 and Q₇ and Q₈ are alkyl, aralkyl, heteroaralkyl, aryl, heteroaryl, alkanoyl, aroyl, aralkanoyl, heteroaralkanoyl, or heteroaroyl in which the alkyl portions can be cyclic and can contain from 1 to 3 heteroatoms which can be N, O, or S, and when Q₁ and Q₂ are present together and are alkyl, they can be taken together to form a 5 or 6 member ring which may contain 1 other heteroatom which can be N, O, or S, of which the N may be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

Where Q₇ and Q₈ can be taken together to form a five or six member ring, the ring is typically pyrrolidine, piperidine, or morpholine. The pyrrolidine ring can be optionally substituted with oxo. The piperidine ring can be optionally substituted with methyl or ethyl. Typically, p is 2 or 3.

In another preferred example, D is an alkylpyrrolidine moiety of the structure

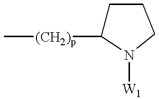

wherein p is an integer from 1 to 6 and W₁ is selected from the group consisting of methyl, ethyl, and propyl. Typically, W₁ is methyl. Typically, p is 2.

Preferably, a compound useful in methods according to the present invention has a log P of from about 1 to about 4 in order to optimize bioavailability and CNS penetration of the compound.

Exemplary studies and treatments were performed as discussed below using various dosages and routes of administration of selected exemplary purine derivatives representative of compositions that are effective with the methods of the present invention. Of course, those skilled in the art will recognize that the present invention is not specifically limited to the particular compositions, dosages or routes of administration detailed below.

Depending upon the particular needs of the individual subject involved, the compositions used in the present invention may be administered in various doses to provide effective treatment concentrations based upon the teachings of the present invention. What constitutes an effective amount of the selected composition will vary based upon such factors including the activity of the selected compound, the physiological characteristics of the subject, the extent and nature of the subject's disease or condition and the method of administration. Exemplary treatment concentrations which have proven effective in modifying neural activity range from less than 1 μM to concentrations of 500 mM or more. Generally, initial doses will be modified to determine the optimum dosage for treatment of the particular mammalian subject. The compositions may be administered using a number of different routes including orally, topically, transdermally, intraperitoneal injection or intravenous injection directly into the bloodstream. Of course, effective amounts of the compounds may also be administered through injection into the cerebrospinal fluid or infusion directly into the brain, if desired.

The methods of the present invention may be effected using compounds administered to a mammalian subject either alone or in combination as a pharmaceutical formulation. Further, the compounds may be combined with pharmaceutically acceptable excipients and carrier materials such as inert solid diluents, aqueous solutions or non-toxic organic solvents. If desired, these pharmaceutical formulations may also contain preservatives and stabilizing agents and the like, as well as minor amounts of auxiliary substances such as wetting or emulsifying agents, as well as pH buffering agents and the like which enhance the effectiveness of the active ingredient. The pharmaceutically acceptable carrier can be chosen from those generally known in the art, including, but not limited to, human serum albumin, ion exchangers, dextrose, alumina, lecithin, buffer substances such as phosphate, glycine, sorbic acid, potassium sorbate, propylene glycol, polyethylene glycol, and salts or electrolytes such as protamine sulfate, sodium chloride, or potassium chloride. Other carriers can be used.

Liquid compositions can also contain liquid phases either in addition to or to the exclusion of water. Examples of such additional liquid phases are glycerin, vegetable oils such as cottonseed oil, organic esters such as ethyl oleate, and water-oil emulsions.

The compositions can be made into aerosol formations (i.e., they can be “nebulized”) to be administered via inhalation. Aerosol formulations can be placed into pressurized acceptable propellants, such as dichloromethane, propane, or nitrogen. Other suitable propellants are known in the art.

Formulations suitable for parenteral administration, such as, for example, by intravenous, intramuscular, intradermal, and subcutaneous routes, include aqueous and non-aqueous, isotonic sterile injection solutions. These can contain antioxidants, buffers, preservatives, bacteriostatic agents, and solutes that render the formulation isotonic with the blood of the particular recipient. Alternatively, these formulations can be aqueous or non-aqueous sterile suspensions that can include suspending agents, thickening agents, solubilizers, stabilizers, and preservatives. Compositions suitable for use in methods according to the present invention can be administered, for example, by intravenous infusion, orally, topically, intraperitoneally, intravesically, or intrathecally. Formulations of compounds suitable for use in methods according to the present invention can be presented in unit-dose or multi-dose sealed containers, in physical forms such as ampules or vials.

The invention is illustrated by the following Examples. These Examples are presented for illustration only and are not intended to limit the invention.

### EXAMPLE 1

Effect of Administration of the Bifunctional Purine Derivative N-4-Carboxyphenyl-3-(6-Oxohydropurin-9-yl) Propanamide on the Levels of Synaptophysin and sAPP Formation

Alzheimer's disease (AD) is characterized by a severe loss of presynaptic cholinergic neurons and decreased levels of acetylcholine and choline acetyltransferase in the cortex (1). Inhibition of cholinergic activity in the central nervous system (CNS) of patients with AD correlated with deterioration in scores on dementia rating scales. Currently, cholinesterase inhibition is the most widely studied and developed approach for treating symptoms of AD. Because anticholinesterase drugs such as tacrine, donepezil, and rivastigmine only moderately improve symptoms in AD, an alternative cholinergic approach that is not entirely based on cholinesterase inhibition but that improves other known biochemical abnormalities associated with the disease should be tried.

One of the major neurochemical changes in AD is the cortical extracellular and vascular deposition of the amyloid beta-peptide (Aβ) which is derived from a large glycosylated membrane-bound beta-amyloid precursor protein (APP) (2). A constitutively expressed putative α-secretase enzyme bisects the Aβ domain within APP to release carboxyl-truncated soluble derivatives (sAPP) in conditioned media of cells (2). The goal of the work reported in this Example is to determine whether the drug AIT-082 can regulate the levels of Aβ.

AIT-082 is currently being investigated in clinical trials for the treatment of AD. It has been shown that AIT-082 can induce the expression of at least three neurotrophins: nerve growth factor (NGF), neurotrophin-3, and basic fibroblast growth factor (bFGF) (3). A combination of factors has been most effective in producing optimal trophic support for compromised neuron functions (3). However, the effects of AIT-082 and trophic factors on the regulation of sAPP and Aβ have not been clearly explored. It is reasonable to hypothesize that multiple trophic factors may synergistically regulate the processing of sAPP in a way that can lead to lower levels of Aβ. In the results reported in this Example, the level of sAPP in PC12 cells that were treated with NGF or AIT-082 was investigated.

Experimental Procedures

Materials. AIT-082 was obtained from NeoTherapeutics (Irvine, Calif.). Nerve growth factor (NGF) and basic fibroblast growth factor (bFGF) were procured from Life Technologies (Gaithersburg, Md.). Other chemicals were of high purity and purchased from Sigma (St. Louis, Miss.).

Treatment of Cells and Preparation of Cell Extract. PC12 cells were first grown to 70-80% confluence in the regular medium. A day prior to the experiment, PC12 cells were subcultured uniformly onto the plate with minimum cellular aggregation/ clumping to approximately 1×10⁶ cells per 60-mm plate. The PC12 cells were then subjected to treatments with either AIT-082, NGF, bFGF or a combination as previously described (4). AIT-082 was added into separate plates at 11 different doses: 0, 5, 20, 30, 50, 100, 300 ng/ ml and also 1, 3, 10, 30, 100 μg/ml. For comparative purposes, cultures were treated with NGF at 10 and 50 ng/ml, and bFGF was used at 50 ng/ml. Additional cultures contained both AIT-082 (300 ng/ml) and either NGF (50 ng/ml) or bFGF (50 ng/ml). Following incubation for 48 hours, the conditioned medium from each plate was collected.

PAGE and Western Immunoblotting. Total proteins from the conditioned media were analyzed on a 12% polyacrylamide gel containing SDS (SDS-PAGE), and western blot analysis was performed in the Mini-PROTEAN II system of Bio-Rad as described previously (6). sAPP was detected using the 22C11 (Boehringer Mannheim, Indianapolis, Ind.). A biotinylated secondary antibody, horse anti-mouse (Boehringer Mannheim), was also used. The detection system was based on the avidin-biotinylated-complex (Vector labs, Burlingame, Calif.) and enzymatic color reactions.

Results

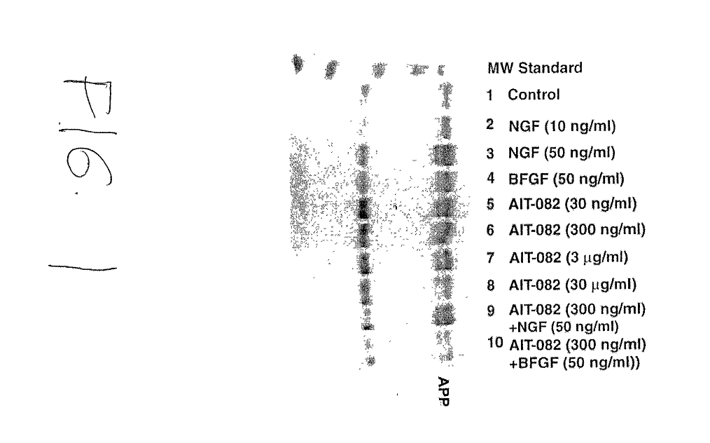

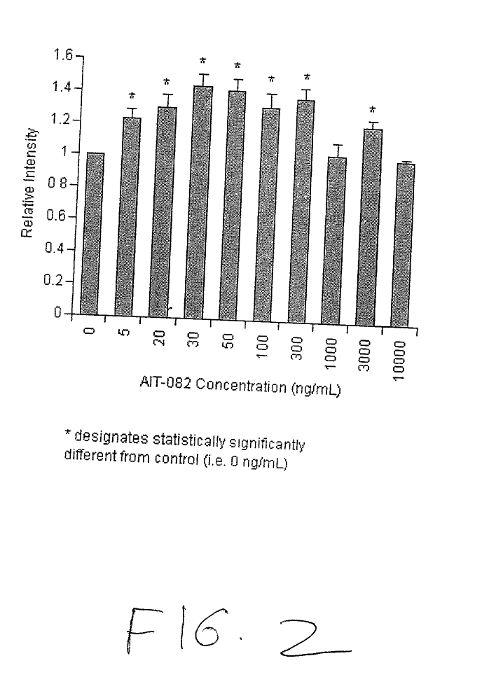

Treatment of NGF or bFGF Results in a Substantial Increase in sAPP Secretion in PC12 Cells. In denaturing polyacrylamide gel, an equal amount of total protein was loaded from each of the conditioned medium samples. After 48 hours of incubation, 50 μg samples of conditioned media were subjected to SDS-PAGE (12%) and transferred to a nitrocellulose membrane. Protein size markers shown on the left of the blot from top to bottom are: 107, 74, 49.3, 36.4, and 28.5 kDa. For FIG. 1, the transferred proteins were probed with anti-APP antibody (22C11), and immunodetection was carried out by the enzymatic color method as described previously (5). Two distinct bands of 110 and 95 kDa were detected, which correspond to soluble APP derivatives (sAPP) arising from different alternate forms of APP and/or their posttranslationally modified derivatives. The results suggest that with the NGF treatment (10 and 50 ng/ml), a significant increase in the secretion of sAPP was observed (FIG. 1). NGF was previously shown to induce the release of sAPP from PC12 cultures (7). With the bFGF treatment, a slight increase in the secretion of sAPP was observed from the control. When the PC12 cells were incubated with different doses of AIT-082, a significant increase in sAPP was observed from the control (FIG. 1, lanes 5-8 vs. lane 1 and FIG. 2). When the cells were simultaneously treated with AIT-082 and NGF, a significant increase in sAPP release was also observed (FIG. 1, lane 9 vs. lane 1), which was more than that in cells treated with either NGF or AIT-082 alone. A similar but smaller synergistic effect of bFGF and AIT-082 treatment was also observed (FIG. 1, lane 10 vs. lane 1). FIG. 2 demonstrates a dose response graph of the extracellular APP levels after culture of cells with increasing levels of AIT-082. Concentrations of AIT-082 from 5-300 ng/ml yield statistically significantly higher levels of extracellular sAPP than control cultures.

References

The following references are referred to in Example 1:

1. R. Becker et al., “Alzheimer's Disease: Molecular Biology to Therapy” (Birkhauser, Boston, 1996).

2. D. J. Selkoe, “Alzheimer's Disease: Genotypes, Phenotype, and Treatment.” Science 275: 630-631 (1997).

3. M. P. Rathbone et al., “AIT-082 as a Potential Neuroprotective and Regenerative Agent in Stroke and Central Nervous System Injury,” Exp. Opin. Invest. Drugs 8: 1255-1262 (1999).

4. D. K. Lahiri et al., “Tacrine Alters the Processing of Beta-Amyloid Precursor Protein in Different Cell Lines,” J. Neurosci. Res. 37: 777-787 (1994).

5. D. K. Lahiri & M. R. Farlow, “Differential Effect of Tacrine and Physostigmine on the Secretion of the Beta-Amyloid Precursor Protein in Cell Lines,” J. Mol. Neurosci. 7: 41-49 (1996).

6. N. R. Marquez-Sterling et al., “Trafficking of Cell Surface-Amyloid Precursor Protein: Evidence that a Sorting Intermediate Participates in Synaptic Vesicle Recycling,” J. Neurosci. 17: 140-151 (1997).

7. L. M. Repolo et al., “Nerve and Epidermal Growth Factors Induce the Release of the Alzheimer Amyloid Precursor from PC12 Cell Cultures,” Biochem. Biophys. Res. Commun. 164: 664-670 (1989).

### EXAMPLE 2

Time Course of sAPP Secretion After Administration of AIT-082 to PC12 Cells

To determine the time course of sAPP secretion after administration of AIT-082 or NGF to PC12 cells, an experiment similar to the experiment of Example 1 was carried out using multiple time points. Five to six million PC12 cells were treated in RPMI 1640 and 0.5% FBS with doses of AIT-082 (10 nM-100 μM). NGF treatment resulted in sympathetic neuronal phenotypes in PC12 cells and cotreatment with AIT-082 enhanced NGF-mediated differentiation. Levels of sAPP in samples from conditioned media and cell lysates were measured by Western immunoblotting with anti-sAPP antibody. When PC12 cells were treated with AIT-082 for 6, 12, 24, 48, or 72 hours, there was an increase in levels of secreted sAPP. The increased sAPP secretion with AIT-082 treatment suggests that this compound may enhance the α-secretase pathway and thereby could potentially decrease the amyloidogenic (amyloid formation) pathway.

### EXAMPLE 3

Effect of AIT-082 on the Amyloid Beta Peptide in PC12 cells

To determine the effect of AIT-082 on Aβ levels, PC12 cells were treated with a high dose of AIT-082 (100 μg/ ml) and the conditioned media was analyzed for Aβ. Levels of total Aβ were measured using a sensitive sandwich ELISA. Experiments were done in triplicate and the values were obtained from known standards run in parallel with the samples as described previously (A. Becher et al., “The Synaptophysin-Synaptobrevin Complex: A Hallmark of Synaptic Vessel Maturation,” J. Neurosci. 19: 1922-1931). Our results suggest that there was a decrease in of secreted Aβ with AIT-082 treatment in PC12 cells as shown in Table 1.

TABLE 1
Effect of AIT-082 on the secretion of Aβ in cultured PC12 cells

| | Levels of total Aβ | Percent change |
|---|---|---|
| Drug Treatment | (fmols/ml) | from controls |
| Control | 169 ± 3 | — |
| AIT-082 | 136 ± 6 | −20% |

Advantages of the Invention

The present invention provides new methods for treating patients with a neurological disease or at risk for a neurological disease. The neurological disease to be treated or prevented can be a neurodegenerative disease, such as, but not limited to, Alzheimer's disease (AD). Alternatively, the neurological disease can be a neurodevelopmental disorder such as, but not limited to, Down's syndrome.

The present invention provides methods for increasing the secretion of sAPP and therefore decreasing the formation of Aβ. These methods can be combined with other treatments such as anticholinesterase treatments.

Although the present invention has been described in considerable detail, with reference to certain preferred versions thereof, other versions and embodiments are possible. Therefore, the scope of the invention is determined by the following claims.

We claim:

1. A method of either inhibiting the formation of Aβ or stimulating the formation of sAPP comprising administering to a patient with a neurological disease or a patient at risk of developing a neurological disease an effective amount of a compound having the activity of either inhibiting the formation of Aβ or stimulating the formation of sAPP, the compound comprising: (1) a moiety A selected from the group consisting of a purine moiety, a purine analogue, a tetrahydroindolone moiety, a tetrahydroindolone analogue, a pyrimidine moiety, and a pyrimidine analogue; (2) a hydrocarbyl moiety L of 1 to 6 carbon atoms that is linked to the moiety A and that can be cyclic, with the hydrocarbyl moiety being optionally substituted with one or more substituents selected from the group consisting of lower alkyl, amino, hydroxy, lower alkoxy, lower alkylamino, lower alkylthio, and oxo; and (3) a moiety B that is linked to the moiety L through a carbonyl group wherein B is —OZ or N(Y₁)—D, where Z is hydrogen, alkyl, aryl, heteroaryl, cycloalkyl, aralkyl, or heteroaralkyl; D is a moiety that promotes absorption of the compound having the activity of either inhibiting the formation of Aβ or stimulating the formation of sAPP; and Y₁ is hydrogen, alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms, which can be N, O, or S.

2. The method of claim 1 wherein the compound having the activity of either inhibiting the formation of Aβ or stimulating the formation of sAPP passes through the blood-brain barrier.

3. The method of claim 1 wherein A is a purine moiety.

4. The method of claim 3 wherein A is a substituted or unsubstituted hypoxanthine moiety.

5. The method of claim 4 wherein L has the structure —(CH₂)ₙ—CONH— where n is an integer from 1 to 6.

6. The method of claim 5 wherein the compound having the activity of either inhibiting the formation of Aβ or stimulating the formation of sAPP is a compound of formula (I) 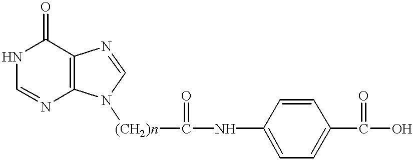
where n is an integer from 1 to 6 and R is hydrogen or lower alkyl or is a salt or prodrug ester of a compound of formula (I) 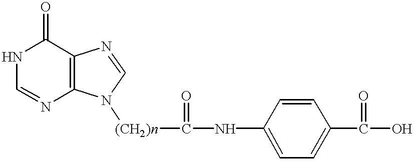
wherein n is an integer from 1 to 6 and R is hydrogen or lower alkyl.

7. The method of claim 6 wherein the compound having the activity of either inhibiting the formation of Aβ or stimulating the formation of sAPP is a compound of formula (I) wherein n is an integer from 1 to 6 and R is hydrogen or lower alkyl.

8. The method of claim 7 wherein R is hydrogen.

9. The method of claim 8 wherein n is 2 and the compound is N-4-[[3-(1,6-dihydro-6-oxopurin-9-yl)-1-oxopropyl]amino]benzoic acid.

10. The method of claim 7 wherein R is ethyl.

11. The method of claim 10 wherein n is 2 and the compound is N4-[[3-(1,6-dihydro-6-oxopurin-9-yl)-1-oxopropyl]amino]benzoic acid ethyl ester.

12. The method of claim 5 wherein the compound having the activity of either inhibiting the formation of Aβ or stimulating the formation of sAPP is a compound of formula (II) 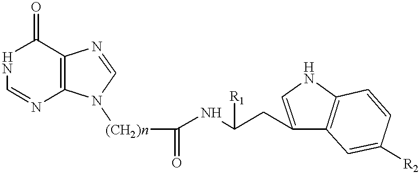
wherein n is an integer from 1 to 6, R is selected from the group consisting of H, COOH, and COOW₁, wherein W₁ is selected from the group consisting of lower alkyl, amino, and lower alkylamino, and R₂ is selected from the group consisting of H and OH.

13. The method of claim 12 wherein n is 2.

14. The method of claim 5 wherein the compound having the activity of either inhibiting the formation of Aβ or stimulating the formation of sAPP is a compound of formula (III) 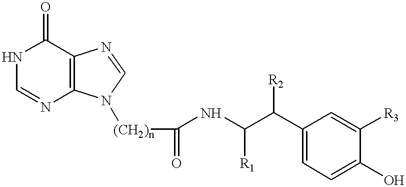
wherein n is an integer from 1 to 6, R₁ is selected from the group consisting of H, COOH, and COOW₁, wherein W₁ is selected from the group consisting of lower alkyl, amino, and lower alkylamino, R₂ is selected from the group consisting of H and OH, and R₃ is selected from the group consisting from the group consisting of H and OH.

15. The method of claim 14 wherein n is 2.

16. The method of claim 3 wherein A is a substituted or unsubstituted guanine moiety.

17. The method of claim 16 wherein L has the structure —(CH₂)ₙ—CONH— wherein n is an integer from 1 to 6.

18. The method of claim 17 wherein the compound having the activity of either inhibiting the formation of Aβ or stimulating the formation of sAPP is a compound of formula (IV) 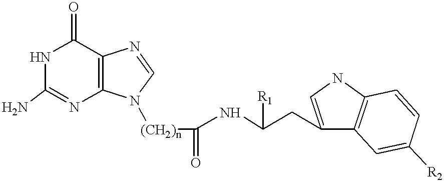
wherein n is an integer from 1 to 6, R₁ is selected from the group consisting of H, COOH, and COOW₁, wherein W₁ is selected from the group consisting of lower alkyl, amino, and lower alkylamino and R₂ is selected from the group consisting of H and OH.

19. The method of claim 18 wherein n is 2, R₁ is H, and R₂ is OH, and the compound is N-(2-(5-hydroxyindol-3-yl))ethyl-3-(2-amino-6-oxohydropurin-9-yl) propanamide.

20. The method of claim 18 wherein n is 2, R₁ is H, and R₂ is H, and the compound is N-(2-(2-indol-3-yl)ethyl))-3-(2-amino-6-oxohydropurin-9-yl) propanamide.

21. The method of claim 18 wherein n is 2, R₁ is COOH, and R₂ is OH, and the compound is N-(1-carboxyl-(2-(5-hydroxyindol-3-yl)ethyl)-3-(2-amino-6-oxohydropurin-9-yl) propanamide.

22. The method of claim 17 wherein the compound having the activity of either inhibiting the formation of Aβ or stimulating the formation of sAPP is a compound of formula (V) 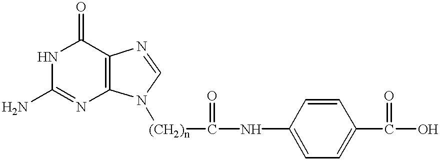
wherein n is an integer from 1 to 6 and R is selected from the group consisting of hydrogen and lower alkyl.

23. The method of claim 22 wherein n is 2, R is hydrogen, and the compound is N-4-carboxyphenyl-3-(2-amino-6-oxohydropurin-9-yl) propanamide.

24. The method of claim 22 wherein n is 2, R is ethyl, and the compound is N-4-carboxyphenyl-3-(2-amino-6-oxohydropurin-9-yl) propanamide ethyl ester.

25. The method of claim 17 wherein the compound having the activity of either inhibiting the formation of Aβ or stimulating the formation of sAPP is a compound of formula (VI) 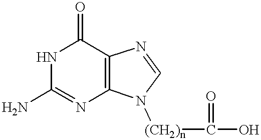
wherein n is an integer from 1 to 6 and R is selected from the group consisting of hydrogen and lower alkyl.

26. The method of claim 25 wherein n is 2, R is hydrogen, and the compound is 3-(2-amino-6-oxohydropurin-9-yl) propanoic acid.

27. The method of claim 25 wherein n is 2, R is ethyl, and the compound is 3-(2-amino-6-oxohydropurin-9-yl) propanoic acid ethyl ester.

28. The method of claim 17 wherein the compound having the activity of either inhibiting the formation of Aβ or stimulating the formation of sAPP is a compound of formula (VII) 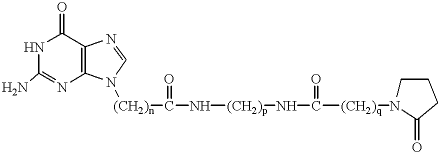
wherein n is an integer from 1 to 6, p is an integer from 1 to 6, and q is an integer from 1 to 3.

29. The method of claim 28 wherein n is 2, p is 2, and q is 1, and the compound is N-[2-[[2-(2-oxopyrrolidin-1-yl)-1-oxoethyl]amino]ethyl]propanamide.

30. The method of claim 17 wherein the compound having the activity of either inhibiting the formation of Aβ or stimulating the formation of sAPP is a compound of formula (VIII) 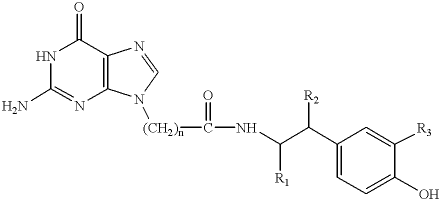
wherein n is an integer from 1 to 6, R₁ is selected from the group consisting of H, COOH, and COOW₁, wherein W₁ is selected from the group consisting of lower alkyl, amino, and lower alkylamino, R₂ is selected from the group consisting of H and OH, and R₃ is selected from the group consisting of H and OH.

31. The method of claim 30 wherein n is 2, R₁ is H, R₂ is H, and R₃ is OH, and the compound is N-(2-(3,4-dihydroxyphenyl)ethyl-3-(2-amino-6-oxohydropurin-9-yl) propanamide.

32. The method of claim 30 wherein n is 2, R₁ is H, R₂ is OH, and R₃ is OH, and the compound is N-(2-hydroxy-2-(3,4-dihydroxyphenyl)ethyl)-3-(2-amino-6-oxohydropurin-9-yl) propanamide.

33. The method of claim 30 wherein n is 2, R₁ is COOH, R₂ is H, and R₃ is H, and the compound is N-(1-carboxyl-2-(3,4-dihydroxyphenyl)ethyl)-3-(2-amino-6-oxohydropurin-9-yl) propanamide.

34. The method of claim 16 wherein the compound having the activity of either inhibiting the formation of Aβ or stimulating the formation of sAPP is a compound of formula (IX) 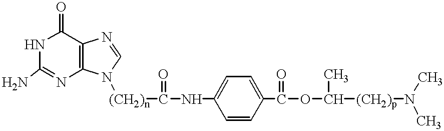
wherein n is an integer from 1 to 6 and p is an integer from 1 to 3.

35. The method of claim 34 wherein n is 2, p is 1, and the compound is N-4-[[3-(2-amino-6-oxohydropurin-9-yl) 1-oxopropyl]amino]benzoic acid 1-(dimethylamino)-2-propyl ester.

36. The method of claim 1 wherein A is a substituted or unsubstituted 9-atom bicyclic moiety in which the 5-membered ring has 1 to 3 nitrogen atoms, the bicyclic moiety having the structure of formula (X) 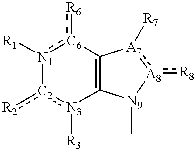
where:
(a) if the bond between N₁ and the bond between C₅ is a single bond, then the bond between C₆ and R₆ is a double bond, R₆ is O or S, and R₁ is hydrogen, alkyl, aralkyl, cycloalkyl, or heteroaralkyl;
(b) if the bond between N₁ and C₆ is a double bond, then the bond between C₆ and R₆ is a single bond, R₁ is not present, and R₆ is hydrogen, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(c) if the bond between C₂ and N₃ is a single bond, then the bond between C₂ and R₂ is a double bond, R₂ is O or S, and R₃ is hydrogen or alkyl;
(d) if the bond between C₂ and N₃ is a double bond, then the bond between C₂ is a single bond, R₃ is not present, and R₂ is hydrogen, alkyl, aralkyl, cycloalkyl, heteroaralkyl, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(e) A₇ and A₈ are C or N;
(i) if A₇ and A₈ are both C and the bond between A₇ and A₈ is a single bond, then the bond between A₈ and R₈ is two single bonds to two hydrogen atoms or is a double bond in which R₈ is O or S and R₇ is two hydrogen atoms;
(ii) if A₇ and A₈ are both C and the bond between A₇ and A₈ is a double bond, then R₇ is hydrogen, the bond between A₈ and R₈ is a single bond and R₈ is hydrogen, halo, alkyl, alkenyl, aryl, aralkyl, aralkenyl, heteroaryl, heteroaralkyl, or heteroaralkenyl;
(iii) if A₇ and A₈ are both N, then the bond between A₇ and A₈ is a double bond, and R₇ and R₈ are not present;
(iv) if A₇ is C and A₈ is N, then the bond between A₇ and A₈ is a double bond, R₇ is hydrogen, and R₈ is not present;
(v) if A₇ is N, A₈ is C, and the bond between A₇ and A₈ is a double bond, then R₇ is not present, the bond between A₈ is a single bond, and R₈ is hydrogen, halo, alkyl, alkenyl, aryl, aralkyl, aralkenyl, heteroaryl, heteroaralkyl, or heteroaralkenyl;
(vi) if A₇ is N, A₈ is C, and the bond between A₇ and A₈ is a single bond, then R₇ is hydrogen, alkyl, aryl, aralkyl, heteroaryl, or heteroaralkyl, the bond between A₈ and R₈ is a double bond, and R₈ is O or S; and
(f) N₉ is bonded to L; with the proviso that A does not have the structure of an unsubstituted guanine or hypoxanthine.

37. The method of claim 3 wherein the purine moiety is a purine moiety of formula (XI) 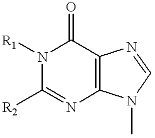
in which:
(a) R₁ is selected from the group consisting of hydrogen, alkyl, aralkyl, cycloalkyl, and heteroaralkyl; and R₂ is selected from the group consisting of hydrogen, alkyl, aralkyl, cycloalkyl, heteroaralkyl, halo, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, arylkoxycarbonyl, heteroarylokoxycarbonyl, alkylaminocarbonyl, arylaminocarbonyl, heteroarylaminocarbonyl, aralkylaminocarbonyl, or heteroarylkylaminocarbonyl in which the alkyl portions could be cyclic and can contain from one to three heteroatoms which could be N, O, or S, with the proviso that both R₁ and R₂ are not hydrogen and that R₁ is not hydrogen when R₂ is amino.

38. The method of claim 37 wherein R₁ is butyl and R₂ is hydrogen.

39. The method of claim 37 wherein R₁ is benzyl and R₂ is hydrogen.

40. The method of claim 37 wherein R₁ is dimethylaminoethyl and R₂ is hydrogen.

41. The method of claim 37 wherein R₁ is cyclopentyl and R₂ is hydrogen.

42. The method of claim 37 wherein R₁ is cyclohexylmethyl and R₂ is hydrogen.

43. The method of claim 37 wherein R₁ is cyclopropylmethyl and R₂ is hydrogen.

44. The method of claim 37 wherein R₁ is hydrogen and R₂ is phenyl.

45. The method of claim 37 wherein R₁ is hydrogen and R₂ is butyl.

46. The method of claim 37 wherein R₁ is butyl and R₂ is butyl.

47. The method of claim 37 wherein R₁ is hydrogen and R₂ is methyl.

48. The method of claim 37 wherein R₁ is hydrogen and R₂ is phenylamino.

49. The method of claim 3 wherein the purine moiety is a purine moiety of Formula (XII) 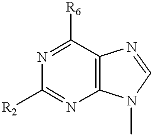
in which:
(a) R₂ is selected from the group consisting of hydrogen, halo, amino, OQ₃, SQ₃, NHNH₂, NHOQ₃, NQ₃Q₄, or NHQ₃, where Q₃ and Q₄ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N, O, or S, and when Q₃ and Q₄ are present together and are alkyl, they can be taken together to form a 5- or 6-membered ring which can contain one other heteroatom which can be N, O, or S, of which the N can be further substituted with Y₃ where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaryloxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S; and
(b) R₆ is selected from the group consisting of hydrogen, halo, amino, OQ₅, SQ₅, NHNH₂, NHOQ₅, NQ₅Q₆, or NHQ₆, where Q₅ and Q₆ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, and heteroaralkylsulfonyl in which the alkyl portions can be cyclic and can contain from 1 to 3 heteroatoms which can be N, O, or S, and when Q₅ and Q₆ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, heteroarylsulfonyl, aralkylsulfonyl, heteroaralkylsulfonyl, alkoxycarbonyl, aryloxycarbonyl, heteroaryloxycarbonyl, arylkoxycarbonyl, heteroarylkoxycarbonyl, alkylaminocarbonyl, arylaminocarbonyl, heteroarylaminocarbonyl, aralkylaminocarbonyl, or heteroaralkylaminocarbonyl in which the alkyl portions can be cyclic and can contain from 1 to 3 heteroatoms which can be N, O, or S.

50. The method of claim 49 wherein R₂ is hydrogen and R₆ is amino.

51. The method of claim 49 wherein R₆ is chloro.

52. The method of claim 49 wherein R₆ is phenylamino.

53. The method of claim 49 wherein R₂ is amino and R₆ is chloro.

54. The method of claim 3 wherein the purine moiety is the purine moiety of Formula (XIII) 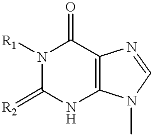
in which:
(a) R₁ is hydrogen, alkyl, aralkyl, cycloalkyl, or heteroaralkyl; and
(b) R₂ is O or S.

55. The method of claim 54 wherein R₁ is hydrogen.

56. The method of claim 54 wherein R₂ is O.

57. The method of claim 54 wherein R₂ is S.

58. The method of claim 3 wherein the compound is 4-[3-(1-benzyl-6-oxo-1,6-dihydropurin-9-yl)propionylamino]benzoic acid ethyl ester.

59. The method of claim 3 wherein the compound is 4-[3-(1-butyl-6-oxo-1,6-dihydropurin-9-yl)propionylamino]benzoic acid ethyl ester.

60. The method of claim 3 wherein the compound is 4-[3-(1-methyl-6-oxo-1,6-dihydropurin-9-yl)propionylamino]benzoic acid ethyl ester.

61. The method of claim 3 wherein the compound is 4-[3-(1-2-dimethylaminoethyl)-6-oxo-1,6-dihydropurin-9-yl) propionylamino]benzoic acid ethyl ester.

62. The method of claim 3 wherein the compound is 4-[3-(2,6-dioxo-1,2,3,6-tetrahydropurin-9-yl) propionylamino]benzoic acid ethyl ester.

63. The method of claim 3 wherein the compound is 4-[3-(6-methoxypurin-9-yl) propionylamino]benzoic acid ethyl ester.

64. The method of claim 3 wherein the compound is 4-[3-(6-dimethylaminopurin-9-yl) propionylamino]benzoic acid ethyl ester.

65. The method of claim 3 wherein the compound is 4-[3-(2-amino-6-chloropurin-9-yl) propionylamino]benzoic acid ethyl ester.

66. The method of claim 3 wherein the compound is 4-[2-(6-oxo-2-thioxo-1,2,3,6-tetrahydropurin-9-yl) propionylamino]benzoic acid ethyl ester.

67. The method of claim 3 wherein the compound is 4-[2-(2-butyl-6-oxo-1,6-dihydropurin-9-yl) propionylamino]benzoic acid ethyl ester.

68. The method of claim 3 wherein the compound is 4-[2-(6-oxo-2-phenyl-1,6-dihydropurin-9-yl) propionylamino]benzoic acid ethyl ester.

69. The method of claim 3 wherein the compound is 4-{[3-(6-chloropurin-9-yl) propionyl]methylamino}benzoic acid methyl ester.

70. The method of claim 3 wherein the compound is 3-(1-benzyl-6-oxo-1,6-dihydropurin-9-yl)-N-[3-(2-oxopyrrolidin-1-yl)propyl]propanamide.

71. The method of claim 3 wherein the compound is 3-(1-benzyl-6-oxo-1,6-dihydropurin-9-yl)-N-{2-[2-(2-oxopyrrolid in-1-yl)acetylamino]ethyl}propanamide.

72. The method of claim 3 wherein the compound is N-[3-(2-oxopyrrolidin-1-yl) propyl]-3-(6-oxo-2-thioxo-1,2,3,6-tetrahydropurin-9-yl)propanamide.

73. The method of claim 3 wherein the compound is 3-(1-benzyl-6-oxo-1,6-dihydropurin-9-yl)-N-(3-morpholin-4-yl)propyl propionamide.

74. The method of claim 1 wherein the compound is a tetrahydroindolone derivative or analogue where A is a 9-atom bicyclic moiety in which the 5-membered ring has one to three nitrogen atoms, the bicyclic moiety having the structure of formula (XIV) 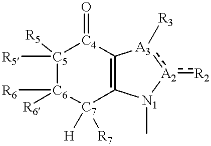
where:
(a) N₁ is bonded to L;
(b) A₂ and A₃ are C or N;
(i) If A₂ and A₃ are both C and the bond between A₂ and A₃ is a single bond, then the bond between A₂ and R₂ is two single bonds, two hydrogen atoms or is a double bond in which R₂ is O or S and R₃ is two hydrogen atoms;
(ii) If A₂ and A₃ are both C and the bond between A₂ and A₃ is a double bond, then R₃ is hydrogen, the bond between A₂ and R₂ is a single bond and R₂ is hydrogen, halo, alkyl, alkenyl, aryl, aralkyl, aralkenyl, heteroaryl, heteroaralkyl, or heteroaralkenyl;
(iii) If A₂ and A₃ are both N, then the bond between A₂ and A₃ is a double bond and R₂ and R₃ are not present;
(iv) If A₂ is N and A₃ is C, then the bond between A₂ and A₃ is a double bond, R₂ is not present, and R₃ is hydrogen;
(v) If A₂ is C, A₃ is N, and the bond between A₂ and A₃ is a double bond, then R₃ is not present, the bond between A₂ and R₂ is a single bond, and R₂ is hydrogen, halo, alkyl, alkenyl, aryl, aralkyl, aralkenyl, heteroaryl, heteroaralkyl, or heteroaralkenyl;
(vi) If A₂ is C, A₃ is N, and the bond between A₂ and A₃ is a single bond, then R₃ is hydrogen, alkyl, aryl, aralkyl, heteroaryl, or heteroaralkenyl, the bond between A₂ and R₂ is a double bond, and A₂ is O or S;
(c) R₅ is hydrogen, alkyl, aryl, aralkyl, heteroaryl, heteroaralkyl, alkanoyl, aroyl, heteroaroyl, aralkanoyl, heteroaralkanoyl, NH₂, NHQ₁, NQ₁Q₂, OH, OQ₁, or SQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom, which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(d) R_{5′} is hydrogen unless R₅ is alkyl, in which case R₅ is hydrogen or the same alkyl as R₅;
(e) R₅ and R_{5′} can be taken together as a double bond to C₅, and can be O, S, NQ₃, or C which can be substituted with one or two groups R₅, where Q₃ is alkyl, aralkyl, heteroaralkyl, aryl, heteroaryl, alkanoyl, aroyl, aralkanoyl, heteroaralkanoyl, or heteroaroyl, in which the alkyl portions can be cyclic and can contain from 1 to 3 heteroatoms which can be N, O, or S;
(f) R₆ is hydrogen, alkyl, aryl, aralkyl, heteroaryl, heteroaralkyl, NH₂, NHQ₄, NQ₄Q₅, OH, OQ₄, or SQ₄, where Q₄ and Q₅ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₄ and Q₅ are present together and are alkyl, they can be taken together to form a 5- or 6-membered ring which can contain one other heteroatom, which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(g) R_{6′} is hydrogen unless R₆ is alkyl, in which case R_{6′} is hydrogen or the same alkyl as R₆;
(h) R₆ and R_{6′} can be taken together as a double bond to C₆ and can be O, S, NQ₆, or C which can be substituted with one or two groups R₅, and where Q₆ is alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S; and
(i) R₇ is hydrogen unless R₅ is alkyl and R_{5′} is hydrogen, in which case R₇ is the same alkyl as R₅.

75. The method of claim 74 wherein A is a tetrahydroindolone moiety.

76. The method of claim 75 wherein the tetrahydroindolone moiety is a tetrahydroindolone moiety of formula (XV) 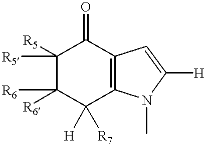
in which:
(a) R₅ is hydrogen, alkyl, aryl, aralkyl, heteroaryl, heteroaralkyl, alkanoyl, aroyl, heteroaroyl, aralkanoyl, heteroaralkanoyl, NH₂, NH₁, NQ₁Q₂, OH, OQ₁, or SQ₁, where Q₁ and Q₂ are alkyl, aralkyl, heteroaralkyl, aryl, heteroaryl, alkanoyl, aroyl, aralkanoyl, heteroaralkanoyl, or heteroaroyl, in which the alkyl portions can be cyclic and can contain from one to three heteroatoms which can be N, O, or S;
(b) R_{5′} is hydrogen;
(c) R₆ is hydrogen, alkyl, aryl, aralkyl, heteroaryl, heteroaralkyl, alkanoyl, aroyl, heteroaroyl, aralkanoyl, heteroaralkanoyl, NH₂, NHW₁, NQ₁Q₂, OH, OQ₁, or SQ₁, where Q₁ and Q₂ are aralkyl, heteroaralkyl, aryl, heteroaryl, alkanoyl, aroyl, aralkanoyl, heteroaralkanoyl, or heteroaroyl, in which the alkyl portions can be cyclic and can contain from one to three heteroatoms which can be N, O, or S and where W₁ is alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from one to three heteroatoms which can be N, O, or S;
(d) R_{6′} is hydrogen; and
(e) R₇ is hydrogen.

77. The method of claim 76 wherein R₅, R_{5′}, R₆, R_{6′}, and R₇ are all hydrogen.

78. The method of claim 77 wherein the compound is 4-[3-(4-oxo-4,5,6,7-tetrahydroindolon-1-yl) propionylamino]benzoic acid ethyl ester.

79. The method of claim 77 wherein the compound is 4-[3-(4-oxo-4,5,6,7-tetrahydroindolon-1-yl) propionylamino]benzoic acid.

80. The method of claim 1 wherein A is an amino-substituted 6-membered heterocyclic moiety of formula (XVI) 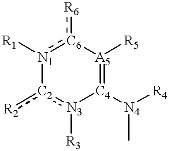
where:
(a) if the bond between N₁ and the bond between C₆ is a single bond, then the bond between C₆ and R₆ is a double bond, R₆ is O or S, and R₁ is hydrogen, alkyl, aralkyl, cycloalkyl, or heteroaralkyl;
(b) if the bond between N₁ and C₆ is a double bond, then the bond between C₆ and R₆ is a single bond, R₁ is not present, and R₆ is hydrogen, halo, amino, OH, OQ₁, SQ₁, NHNH₂, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(c) if the bond between C₂ and N₃ is a single bond, then the bond between C₂ and R₂ is a double bond, R₂ is O or S, and R₃ is hydrogen or alkyl;
(d) if the bond between C₂ and N₃ is a double bond, then the bond between C₂ and R₂ is a single bond, R₃ is not present, and R₂ is hydrogen, alkyl, aralkyl, cycloalkyl, heteroaralkyl, halo, amino, OH, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl, in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain one other heteroatom which can be N, O, or S, of which the N can be further substituted with Y₃, where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(e) R₄ is hydrogen, alkyl, aralkyl, heteroaralkyl, alkanoyl, aroyl, heteroaroyl, aralkanoyl, heteroaralkanoyl, alkylsulfonyl, arylsulfonyl, heteroarylsulfonyl, alkoxycarbonyl, aryloxycarbonyl, heteroaryloxycarbonyl, alkylaminocarbonyl, arylaminocarbonyl, or heteroarylaminocarbonyl;
(f) A₅ is carbon or nitrogen;
(g) if A₅ is nitrogen, then R₅ is not present;
(h) if A₅ is carbon, then R₅ is hydrogen, amino, alkyl, alkoxy, halo, nitro, aryl, cyano, alkenyl, or alkaryl;
(i) if R₅ and R₆ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S; and
(j) N₄ is bonded to L.

81. The method of claim 80 wherein A₅ is carbon and the 6-membered heterocyclic moiety is a pyrimidine moiety.

82. The method of claim 81 wherein R₂ is O and R₃ is hydrogen.

83. The method of claim 82 wherein the pyrimidine moiety is selected from the group consisting of cytosine, thymine, uracil, 3-methyluracil, 3-methylthymine, 4-methylcytosine, 5-methylcytosine, 5-hydroxymethylcytosine, 5-hydroxyuracil, 5-carboxymethyluracil, and 5-hydroxymethyluracil.

84. The method of claim 81 wherein R₂ is S and R₃ is hydrogen.

85. The method of claim 84 wherein the pyrimidine moiety is selected from the group consisting of 2-thiouracil, 5-methylamino-2-thiouracil, 5-methyl-2-thiouracil, and 2-thiocytosine.

86. The method of claim 81 wherein R₂ is amino and the bond between C₂ and N₃ is a double bond.

87. The method of claim 86 wherein the pyrimidine moiety is selected from the group consisting of 2-aminopyrimidinone and 2-amino-4-chloropyrimidine.

88. The method of claim 81 wherein R₂ is hydrogen and the bond between C₂ and N₃ is a double bond.

89. The method of claim 88 wherein the pyrimidine moiety is selected from the group consisting of 4-chloropyrimidine, 5-amino-4-chloropyrimidine, 4-chloro-5-methylpyrimidine, 4-chloro-5-hydroxymethylpyrimidine, and 4-chloro-5-carboxymethylpyrimidine.

90. The method of claim 81 wherein R₁ is hydrogen, methyl, or ethyl, R₅ is hydrogen, methyl, or ethyl, and R₆ is O.

91. The method of claim 90 wherein the pyrimidine moiety is pyrimidinone.

92. The method of claim 81 wherein the compound is 4-[3-(2-amino-6-chloropyrimidin-4-ylamino) propionylamino]benzoic acid ethyl ester.

93. The method of claim 81 wherein the compound is 4-[3-(5-amino-6-chloropyrimidin-4-ylamino) propionylamino]benzoic acid ethyl ester.

94. The method of claim 81 wherein the compound is 4-[3-(6-chloropyrimidin-4-ylamino) propionylamino]benzoic acid ethyl ester.

95. The method of claim 81 wherein the compound is 4-[3-(2-amino-6-chloropyrimidin-4-ylamino) propionylamino]benzoic acid.

96. The method of claim 81 wherein the compound is 4-[3-(6-chloropyrimidin-4-ylamino) propionylamino]benzoic acid.

97. The method of claim 81 wherein the compound is 4-[3-(5-amino-6-chloropyrimidin-4-ylamino) propionylamino]benzoic acid.

98. The method of claim 81 wherein the compound is 3-[3-(2-amino-6-chloropyrimidin-4-ylamino) propionylamino]benzoic acid ethyl ester.

99. The method of claim 81 wherein the compound is 3-[3-(6-chloropyrimidin-4-ylamino) propionylamino]benzoic acid ethyl ester.

100. The method of claim 81 wherein the compound is 3-[3-(5-amino-6-chloropyrimidin-4-ylamino) propionylamino]benzoic acid ethyl ester.

101. The method of claim 81 wherein the compound is 3-[3-(2-amino-6-chloropyrimidin-4-ylamino) propionylamino]benzoic acid.

102. The method of claim 81 wherein the compound is 3-[3-(6-chloropyrimidin-4-ylamino) propionylamino]benzoic acid.

103. The method of claim 81 wherein the compound is 3-[3-(5-amino-6-chloropyrimidin-4-ylamino) propionylamino]benzoic acid.

104. The method of claim 1 wherein L has the structure —(CH₂)ₙ— wherein n is an integer from 1 to 6.

105. The method of claim 104 wherein n is 2.

106. The method of claim 104 wherein n is 3.

107. The method of claim 1 wherein the moiety B is —OZ.

108. The method of claim 107 wherein Z is hydrogen.

109. The method of claim 107 wherein Z is alkyl.

110. The method of claim 109 wherein Z is selected from the group consisting of methyl, ethyl, butyl, propyl, and isopropyl.

111. The method of claim 1 wherein B is —N(Y₁)—D.

112. The method of claim 111 wherein Y₁ is hydrogen.

113. The method of claim 111 wherein Y₁ is lower alkyl.

114. The method of claim 113 wherein Y₁ is methyl.

115. The method of claim 111 wherein D is a moiety having at least one polar, charged, or hydrogen-bond-forming group to increase the water-solubility of the compound.

116. The method of claim 115 wherein D is a carboxylic acid or carboxylic acid ester with the structure 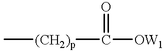
wherein p is an integer from 1 to 6 and W₁ is selected from the group consisting of hydrogen and lower alkyl.

117. The method of claim 116 wherein W₁ is hydrogen.

118. The method of claim 116 wherein W₁ is ethyl.

119. The method of claim 115 wherein D and Y₁ are taken together to form a piperazine derivative of the structure 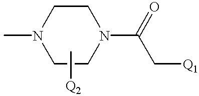
wherein Q₁ is hydrogen, methyl, ethyl, butyl, or propyl, and Q₂ is hydrogen or methyl, where, if Q₂ is methyl, it can be located on either of the two possible positions in the piperazine ring.

120. The method of claim 115 wherein D has the structure 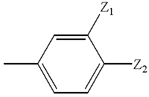
wherein one of Z₁ and Z₂ is hydrogen and the other is Z₁ and Z₂ is —COOH or —COOW₁, wherein W₁ is alkyl.

121. The method of claim 120 wherein W₁ is selected from the group consisting of methyl, ethyl, propyl, butyl, and isobutyl.

122. The method of claim 115 wherein D is a phenylsulfonamidyl moiety of the structure 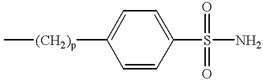
wherein p is an integer from 0 to 6.

123. The method of claim 115 wherein D is an alkylpyridyl moiety of the structure 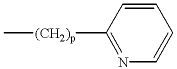
wherein p is an integer from 1 to 6.

124. The method of claim 114 wherein D is an dialkylaminoalkyl moiety of the structure 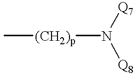
wherein p is an integer from 1 to 6 and Q₇ and Q₈ are alkyl, aralkyl, heteroaralkyl, aryl, heteroaryl, alkanoyl, aroyl, aralkanoyl, heteroaralkanoyl, or heteroaroyl, in which the alkyl portions can be cyclic and can contain from 1 to 3 heteroatoms which can be N, O, or S, and when Q₇ and Q₈ are present together and are alkyl, they can be taken together to form a 5- or 6-membered ring which can contain one other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

125. The method of claim 124 wherein Q₇ and Q₈ are each alkyl.

126. The method of claim 125 wherein Q₇ and Q₈ are each selected from the group consisting of methyl, ethyl, propyl, butyl, and isobutyl.

127. The method of claim 126 wherein Q₇ and Q₈ are taken together to form 5- or 6-membered optionally substituted ring.

128. The method of claim 127 wherein the ring is a morpholinyl ring.

129. The method of claim 127 wherein the ring is a pyrrolidinyl ring that is optionally substituted with oxo.

130. The method of claim 126 wherein the ring is a piperidinyl ring that is optionally substituted with methyl or ethyl.

131. The method of claim 115 wherein D is an alkylpyrrolidinyl moiety of the structure 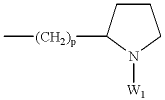
wherein p is an integer from 1 to 6 and W₁ is selected from the group consisting of methyl, ethyl, and propyl.

132. The method of claim 1 wherein the compound has a log P of from about 1 to about 4.

133. The method of claim 1 wherein the neurological disease is a neurodegenerative disease.

134. The method of claim 133 wherein the neurodegenerative disease is Alzheimer's disease.

135. The method of claim 1 wherein the neurological disease is a neurodevelopmental disorder.

136. The method of claim 135 wherein the neurodevelopmental disorder is Down's syndrome.
